(19) United States
(12) Patent Application Publication
(10) Pub. No.: US 2017/0305646 A1
(43) Pub. Date: Oct. 26, 2017

(54) DISPENSING CONTAINER

(71) Applicant: TRIG1 LIMITED, Waterlooville (GB)

(72) Inventors: Raymond John Bacon, Waterlooville (GB); Stuart William Pearce, Blackburn, Lancashire (GB); Alex William Noble, Blackburn, Lancashire (GB)

(21) Appl. No.: 15/520,457

(22) PCT Filed: Oct. 20, 2015
(86) PCT No.: PCT/GB2015/053104

§ 371 Date: Apr. 20, 2017

(30) Foreign Application Priority Data
Oct. 20, 2014 (GB) 1418592.0

(51) Int. Cl. B65D 83/38; B65D 1/02; A61M 15/00; B65D 23/00; G01F 23/00; A61J 1/18

(52) U.S. Cl. CPC B65D 83/38; G01F 23/0007; B65D 1/023; A61J 1/18; B65D 23/001; A61M 15/0068; A61M 15/009; A61J 2205/30; B65D 2203/045

(57) ABSTRACT

The present invention provides a dispensing container comprising a vessel, the vessel having: a formation and/or discrete element in or on the vessel which causes the centre of gravity of contents of the vessel to be positioned differently, when oriented in a lain-on-side attitude, from its notional position, in the absence of the formation or element in or on the vessel; an additional formation or discrete element in or on the vessel which, when the container is in the lain-on-side attitude, lifts at least a portion of the container away from a surface, wherein, when the container is in the lain-on-side attitude and has a first volume of content, the centre of gravity of the content biases the container to a first orientation, and when the container is in the lain-on-side attitude and has a second volume of content, the centre of gravity of the container biases the container to a second orientation, different to the first.

### FIELD

The present invention relates to a dispensing container particularly, but not exclusively, a dispensing container for storing and dispensing medicine.

### BACKGROUND

Dispensing containers come in many shapes and sizes. A particular problem associated with pressurised liquid dispensing containers, but not limited thereto, is the difficulty of determining the level of liquid present within the container. Such containers typically comprise a valve for dispensing liquid from the container and a vessel containing the liquid under pressure. The vessel is typically manufactured from impact extruded aluminium or rolled and welded sheet steel. Such materials are opaque making it impossible to see the quantity of liquid in the container. The quantity of liquid in the container also cannot easily be determined by handling, feel or sound of the container.

This problem is particularly evident in the medical field and particular in relation to inhalers used for respiratory conditions such as asthma. It is critical that a person suffering from a respiratory condition has access to inhalers to relieve symptoms such as being short of breath. Not being able to assess the liquid volume of a medicinal product in an inhaler container is problematic as the user of an inhaler would not be able to identify when they need to order a new inhaler or assess whether the inhaler has dispensed the number of doses that it contains. Thus it is not uncommon for person's suffering from respiratory conditions to either run out of their inhaler before they order a new one or order a new one and dispose of the old inhaler before it is empty thus wasting the contents.

WO2011/095761 provides solutions to the problem of not being able to determine the quantity of liquid in a pressurised liquid dispensing container.

The present invention seeks to provide an improved dispensing container for determining the quantity of liquid therein.

### SUMMARY

The present invention provides a dispensing container comprising a vessel, the vessel having: a formation and/or a discreet element in or on the vessel which causes the centre of gravity of liquid contents of the vessel to be positioned differently, when oriented in a lain-on-side attitude, from its notional position, in the absence of the formation and/or discreet element; and, an additional formation or discrete element in or on the vessel which, when the container is in the lain-on-side attitude, lifts at least a portion of the container away from a surface, wherein, when the container is in the lain-on-side attitude and has a first volume of content, the centre of gravity of the content biases the container to a first orientation, and when the container is in the lain-on-side attitude and has a second volume of content, the centre of gravity of the container biases the container to a second orientation, different to the first.

The provision of an additional formation or discrete element in or on the vessel to lift the vessel away from a surface when oriented in a lain-on-side attitude permits the container to roll under the influence of the centre of gravity of the container and/or any content therein even if the container encounters debris such as food crumbs, grit or hair, for example.

The additional formation or discreet element in or on the vessel for lifting the container away from a surface may take the form of at least one circumferential band, or tyre. Preferably, the at least one circumferential band, or tyre, comprises two circumferential bands or tyres. Extends around a portion of container

Use of an additional discreet element on the vessel, such as a circumferential band or tyre would enable such a band, or tyre, to be retrospectively installed to containers after manufacture.

The band, or tyre, may be positioned within a circumferential groove running around the vessel of the container.

The additional formation or discreet element in or on the vessel may alternatively take the form of an integral circumferential protuberance from the vessel or a 3D printed, or embossed logo.

The additional formation or discreet element in or on the vessel may further comprise an outwardly facing projection for preventing the container from settling in a certain orientation. The projection introduces instability into the container such that the centre of gravity of the content or the container will bias the container in the first orientation or second orientation respectively overcoming equilibrium in the case the centre of gravity of the content and centre of gravity of the container are equal.

### BRIEF DESCRIPTION OF THE DRAWINGS

Certain embodiments will now be described with reference to the following drawings:

FIG. 1 shows a side view of a roll-stable container with an external groove positioned beneath the level of liquid therein;

FIG. 2 shows a side view of the roll stable container of FIG. 1 with the groove positioned above the level of liquid therein;

FIG. 3 shows a side view of the roll stable container of FIGS. 1 and 2 with the groove effectively floating on the surface of the liquid therein;

FIG. 4a shows a side view of a first roll stable container in accordance with the invention;

FIG. 4b shows a detail view of part of the first roll stable container of FIG. 4a;

FIG. 5a shows a side view of a second roll stable container in accordance with the invention;

FIG. 5b shows a detail view of part of the second roll stable container of FIG. 5a;

FIG. 6a shows a side view of a third roll stable container in accordance with the invention;

FIG. 6b shows a detail view of part of the third roll stable container of 6a.

### DETAILED DESCRIPTION OF THE CERTAIN EMBODIMENTS

The certain embodiments will now be described, by way of example only, with reference to the brief description of the drawings.

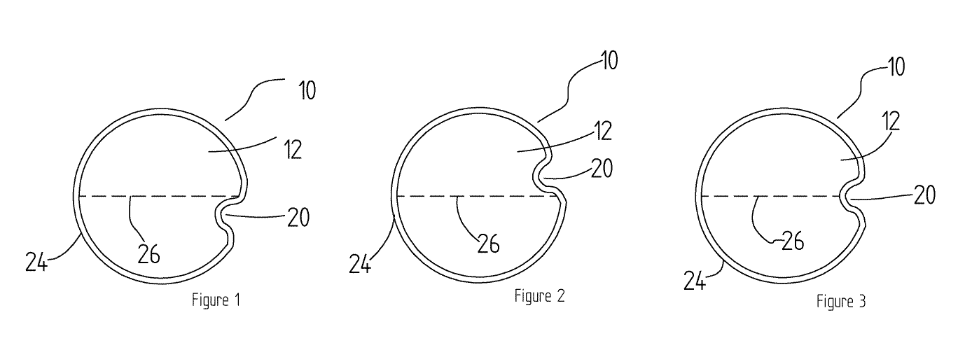

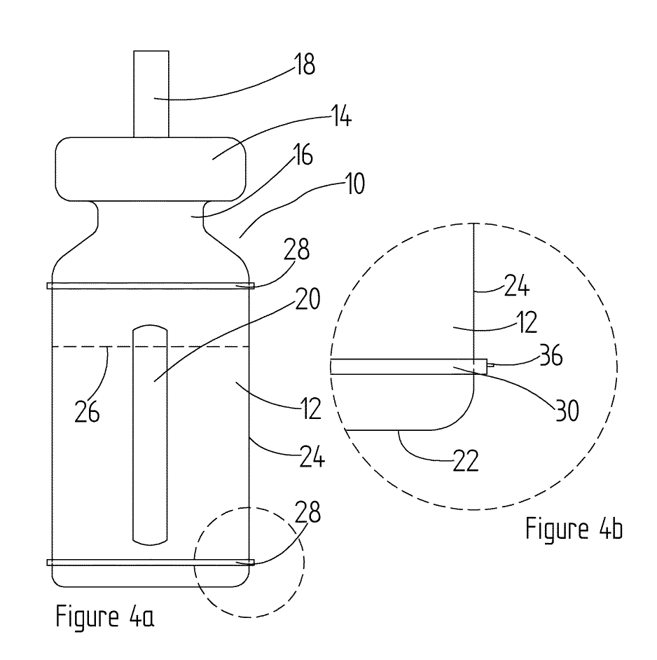

Referring to the figures, the dispenser 10 thereshown comprises a vessel 12 with a valve 14 crimped on at a neck 16 in the vessel. The valve has a dispensing stem 18. The vessel is circularly cylindrical and formed with a groove 20 within its circular cylindrical envelope, extending parallel with the length of the vessel giving it an inflected cylindrical sidewall. The arrangement is such that when the container is in an attitude of being lain on its sidewall 24, in which the groove 20 is, if the groove 20 is initially beneath the surface of the liquid 26 in the container, as shown in FIG. 1, the container 10 will roll, changing its orientation, until the groove 20 is at the surface of the liquid 26, as shown in FIG. 3, with the groove 20 in effect floating on the surface of the liquid 26. If the groove 20 is initially above the liquid 26, as shown in FIG. 2, since it biases the centre of gravity of the vessel 12 and the container 10 towards itself, being of constant wall thickness or having a bias weight in or on the vessel, the container 10 will roll again until the groove 20 is floating on the surface of the liquid 26, as shown in FIG. 3. Thus the extent of fullness of the container 10 can be determined by where the groove 20 comes to rest.

As shown in the figures, the groove 20 may extend only partially along the length of the vessel 12. The groove 20 may be oriented centrally in the longitudinal dimension of the vessel 12 or offset. In other embodiments the groove 20 may extend along the entire longitudinal dimension of the vessel from a base 22 to the neck 16.

When the liquid level is low, the groove 20 extending from end to end of the vessel 12 provides a particularly accurate indication of content, since liquid 26 cannot flow behind it.

To assist the container 10 in being able to roll, a substantially circumferential formation 28 is provided at least at one end of the vessel 12 to lift the sidewall 24 of the vessel 12 away from a surface. FIGS. 4a and 4b show a roll-stable pressurised liquid dispenser 10 which has a tyre 30, or band, positioned around the vessel 12 at each end thereof. The tyre 30, or band, has a width of approximately 1 mm and a depth of approximately 1 mm. The tyre 30, or band, lifts the sidewall 24 of the vessel away from a surface by a distance of approximately 1 mm. The tyre 30, or band, can be made from rubber, silicone, plastic, metal, or any other suitable material. The tyre 30, or band, has a smooth surface so as to reduce friction between the tyre 30, or band, and a surface. The formation 28 may extend around the entire circumference of the vessel 12 or only partially around the circumference of the vessel 12.

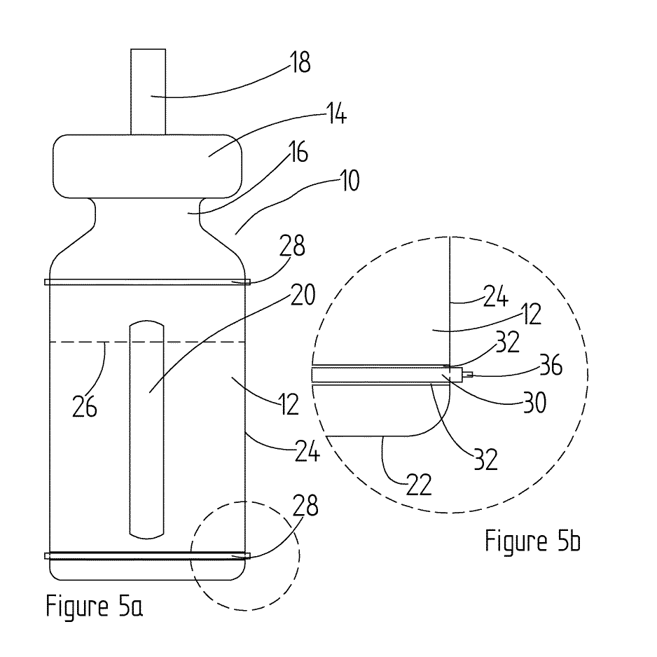

The tyre 30, or band, can be adhered to the vessel 12 or can utilise the elastic properties of the tyre 30, or band, to hold the tyre 30, or band, in the desired position. In some embodiments of the invention, the tyre 30, or band, can be positioned within a circumferential groove 32 in the sidewall 24 of the vessel 12, as shown in FIGS. 5a and 5b.

In certain embodiments the formation 28 may be part of a label which is embossed or printed, for example to provide a raised area.

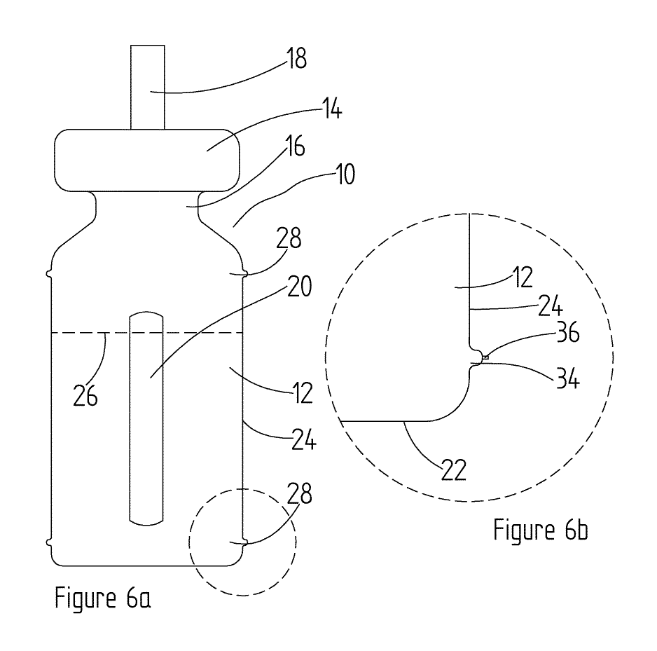

FIGS. 6a and 6b show a dispenser 10 which has an integral circumferential protuberance 34 formed in the sidewall 24 of the vessel 12. The protuberance defines a substantially uniformly curved surface which protrudes approximately 1 mm from the sidewall 24 of the vessel 12.

As shown in FIGS. 4b, 5b and 6b, the formation 28 is provided with a projection 36 which projects outwardly from the formation 28 to introduce an element of instability to the container. The projection 36 prevents the container 10 from settling at a dead centre orientation. In one embodiment the projection is a pin which has a length of between 1 mm and 10 mm and a width of between 0.5 mm and 3 mm. The pin is made from the same material as the circumferential protuberance, tyre or 3D printed label and is integral therewith. However, the skilled person will appreciate that in other embodiments the projection may be a separate component from the circumferential protuberance 34, tyre 30 or 3D printed label and maybe formed from a different material. The projection, in some embodiments, may also be a protuberance, spike or bump, for example.

1. A dispensing container comprising a vessel, the vessel having:
a formation and/or discrete element in or on the vessel which causes the center of gravity of liquid contents of the vessel to be positioned differently, when oriented in a lain-onside attitude, from its notional position, in the absence of the formation or element;
an additional formation or discrete element in or on the vessel which, when the container is in the lain-on-side attitude, lifts at least a portion of the container away from a surface,
wherein, when the container is in the lain-on-side attitude and contains a first volume of content, the center of gravity of the content biases the container to a first orientation, and
when the container is in the lain-on-side attitude and has a second volume of content, the center of gravity of the container biases the container to a second orientation, different to the first.

2. A dispensing container according to claim 1, wherein the additional formation or element in or on the vessel which, when the container is in the lain-on-side-attitude, lifts the container away from a surface comprises at least one tire, or band, arranged circumferentially around the vessel.

3. A dispensing container according to claim 1, wherein the additional formation or element in or on the vessel which, when the container is in the lain-on-side-attitude, lifts the container away from a surface comprises at least one integral circumferential protuberance.

4. A dispensing container according to claim 1, wherein the formation or element in or on the vessel which, when the container is in the lain-on-side-attitude, lifts the container away from a surface comprises a 3D printed, or embossed, label.

5. A dispensing container according to claim 2, wherein the at least one tire, or band, comprises two tires, or bands.

6. A dispensing container according to claim 5, wherein one tire or band is positioned adjacent a base wall of the vessel and the other tire, or band, is positioned adjacent a neck portion of the vessel.

7. A dispensing container according to claim 5, wherein each tire, or band, is positioned within a circumferential groove in a side wall of the vessel.

8. A dispensing container according to claim 5, wherein each tire, or band, is affixed to a sidewall of the vessel by adhesive.

9. A dispensing container according to claim 5, wherein each tire, or band, is manufactured from rubber, silicone, plastic, metal, or any other suitable material.

10. A dispensing container according to claim 5, wherein each tire, or band, has a width of approximately 1 mm and a depth of approximately 1 mm.

11. A dispensing container according to claim 2, wherein the at least one integral circumferential protuberance comprises two integral circumferential protuberances.

12. A dispensing container according to claim 11, wherein each integral circumferential protuberance defines a substantially uniform curved surface.

13. A dispensing container according to claim 11, wherein each integral circumferential protuberance has a width of approximately 1 mm and a depth of approximately 1 mm.

14. A dispensing container according to claim 11, wherein one integral circumferential protuberance is positioned adjacent a base wall of the vessel and wherein the other integral circumferential protuberance is positioned adjacent a neck portion of the vessel.

15. A dispensing container according to claim 1, wherein the additional formation or discrete element in or on the vessel comprises an outwardly facing projection.

16. A dispensing container according to claim 15, wherein the outwardly facing projection comprises a pin.

17. A dispending container according to claim 15, wherein the outwardly facing projection has a length of less than two millimeter and a width of less than 1 millimeter.

18. (canceled)
